SEQUENCE LISTING (2 sequences)

SEQ ID NO: 1 - DNA, 9110 bp, Homo sapiens
gatgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgacccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagcccaccagtgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccagctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacagggcctccctggcccctctggtccccctggattaccgggaaagccaggcgaggatggcaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactgaggcccagataatgagctgagattcagcatcccctggaggagtcggggtctcagcagaaccccactgtccctccccttggtgctagaggcttgtgtgcacgtgagcgtgcgtgtgcacgtccgttatttcagtgacttggtcccgtgggtctagccttcccccctgtggacaaacccccattgtggctcctgccaccctggcagatgactcactgtgggggggtggctgtgggcagtgagcggatgtgactggcgtctgacccgccccttgaccc

SEQ ID NO: 2 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

(19) United States
(12) Patent Application Publication
(10) Pub. No.: US 2018/0036366 A1
(43) Pub. Date: Feb. 8, 2018

(54) ADMINSTRATION OF RECOMBINANT COLLAGEN 7 FOR THE TREATMENT OF AGE RELATED DISORDERS

(71) Applicant: PHOENIX TISSUE REPAIR, INC., ANDOVER, MA (US)

(72) Inventor: Barbara Gilchrest, Boston, MA (US)

(21) Appl. No.: 15/791,577

(22) Filed: Oct. 24, 2017

Related U.S. Application Data
(62) Division of application No. 14/655,251, filed on Jun. 24, 2015, now abandoned.
(60) Provisional application No. 61/746,421, filed on Dec. 27, 2012.

(51) Int. Cl. A61K 38/13; A61K 38/19; A61K 38/39; A61K 38/21

(52) U.S. Cl. CPC A61K 38/13; A61K 38/39; A61K 38/212; A61K 38/191; A61K 2300/00

(57) ABSTRACT

The present disclosure provides methods of treating age related disorders through the administration of collagen 7 or the functional fragment or variant thereof.

### CROSS REFERENCE TO RELATED APPLICATIONS

This application is a Divisional application of U.S. patent application Ser. No. 14/655,251 filed on Jun. 24, 2015, which is a U.S. National Stage application of PCT/US2013/077479, filed Dec. 23, 2013, which claims the benefit of U.S. Provisional application No. 61/746,421, filed Dec. 27, 2012. The contents of each of which are herein incorporated by reference in their entirety.

### REFERENCE TO SEQUENCE LISTING

The present application is being filed along with a Sequence Listing in electronic format. The Sequence Listing is provided as a file entitled SEQLST2118-1005USDIV.txt, created on Oct. 24, 2017, which is 38,271 bytes in size. The information in the electronic format of the sequence listing is incorporated herein by reference in its entirety.

### BACKGROUND OF THE INVENTION

Collagen 7 functions to strengthen and stabilize the skin, in that it is a major component of anchoring fibrils (“AFs”), which help anchor the top layer of the skin, the epidermis, to the underlying dermis. Chronically sun-exposed, photodamaged or photoaged skin is relatively fragile compared to sun-protected skin. This fragility has been attributed at least in part to decreased number of anchoring fibrils in the damaged skin caused by decreased collagen 7 mRNA expression in the damaged skin (Woodley et al. (1990) JAMA 263(22): 3057-3059; Craven et al. (1997) British Journal of Dermatology 137: 344-350). In addition to regulation of collagen 7 expression at the mRNA level as described above, collagen 7 expression can also be regulated through the expression and activity of collagenases. Collagenases are enzymes that catalyze the break down of collagens through hydrolysis of the peptide bonds in triple helical regions of collagen proteins. Collagen 7 can be reduced at DEJ in areas of skin that are malignant or areas of high statistical risk of skin cancer, e.g., shot exposed skin, aged skin, sun exposed skin; and this may facilitate the intervention. Chronically sun-exposed and sun-protected older skin have also been associated with increased levels of collagenase activity relative to younger skin (Orringer el al. 2004 Arch Dermatol 140: 1326-1332; Fisher et al. 1997 NEJM 337: 1419-1428).

### SUMMARY OF THE INVENTION

The present disclosure is based, at least in part, on the finding that decreased collagen 7 levels occur in the subjects having various disorders, e.g., such as age-related disorders. For example, it has been found that there is decreased collagen 7 expression in the skin of elderly subjects. Accordingly, the disclosure provides, inter alia, methods of treating an elderly subject, having an age related disorder, through the administration of collagen 7 or functional fragments and variants thereof.

In one aspect, the disclosure features a method of treating a subject, the method comprising: administering to an elderly subject having or at risk of having an age related disorder, collagen 7 or functional fragments and variants thereof.

In certain embodiments, the method further comprises selecting a subject for administration of collagen 7, or functional fragments or variants thereof, based at least in part upon the determination that the subject is an elderly subject. In certain embodiments, the method comprises selecting a subject for administration of collagen 7, or functional fragments or variants thereof, based, at least in part, upon the determination that the subject is elderly and has or is at risk of having an age related disorder.

In certain embodiments, the elderly subject is a subject at least 60 years or over, e.g., over the age of 65, over the age of 70, over the age of 75, over the age of 80, over the age of 85, over the age of 90, over the age of 95, over the age of 100, over the age of 105, over the age of 110, or over the age of 115.

In certain embodiments, the elderly subject is a subject at least 60 years of age, at least 65 years of age, at least 70 years of age, at least 75 years of age, at least 80 years of age, at least 85 years of age, at least 90 years of age, at least 95 years of age, at least 100 years of age, at least 105 years of age, at least 110 years of age, or at least 115 years of age.

In certain embodiments, the elderly subject is between the ages of 60-120, between the ages of 60-110, between the ages of 60-100, between the ages of 60-90, between the ages of 60-80.

In certain embodiments, the elderly subject is between the ages of 60-70, between the ages of 70-80, between the ages of 80-90, between the ages of 90-100, between the ages of 100-110, or between the ages of 110-120.

In certain embodiments, the elderly subject is between the ages of 60-65, between the ages of 65-70, between the ages of 70-75, between the ages of 75-80, between the ages of 80-85, between the ages of 85-90, between the ages of 90-95, between the ages of 95-100, between the ages of 100-105, or between the ages of 105-110, between the ages of 110-115.

In certain embodiments, the subject is below the age 60 and has one or more areas of skin which are prematurely aged, e.g., by habitual sun exposure.

In certain embodiments, the age related disorder is a chronic disorder. In certain embodiments the age related disorder is a cancer, e.g., skin cancer; mucosal tissue tearing, e.g., vaginal or anal tearing; chronic or non-healing wound; or diabetes. In certain embodiments, the age related disorder is a skin cancer e.g., skin cancer, e.g., squamous cell carcinoma; melanoma, e.g., superficial spreading melanoma, nodular melanoma, lentigo malignant melanoma, acral lentiginous melanoma; basal cell carcinoma; or Kaposi's sarcoma.

In certain embodiments, the subject is an elderly subject that is at risk for an age-related disorder. For example, the subject has a previous diagnosis of a skin cancer, e.g., squamous cell carcinoma; melanoma, e.g., superficial spreading melanoma, nodular melanoma, lentigo maligna melanoma, acral lentiginous melanoma; basal cell carcinoma; or Kaposi's sarcoma; the subject is a recipient of an organ transplant, e.g., a solid organ transplant recipient; the subject currently or previously used a tobacco product, e.g., cigarette smoking, cigar smoking, chewing tobacco, dipping or spit tobacco; the subject has a previous or current diagnosis of a chronic or nonhealing wound, e.g., pressure sore, chronic ulcer, or bed sore; the subject has limited mobility; the subject has long term sun exposure, long term daily sun exposure; or a skin type associated with an risk of susceptibility to skin cancer, e.g., skin phototype I, skin phototype II, skin phototype III.

In some embodiments, the collagen 7, or the functional fragment or variant thereof, is administered as a single dose. In some embodiments, the collagen 7, or the functional fragment or variant thereof, is administered as multiple doses (an initial dose and one or more subsequent doses) over a period of time.

In one embodiment, multiple doses of the collagen 7, or the functional fragment or variant thereof, are administered for a period of at least 3 months, 4 months, 5 months, 6 months, 9 months, 12 months, 15 months, 18 months, 21 months, 2 years, three years, four years, five years, six years or more (e.g., over the remaining lifetime, over a lifetime).

In one embodiment, each subsequent dose of collagen 7, or the functional fragment or variant thereof, is administered two weeks, three weeks, four weeks, five weeks, six weeks, eight weeks after the previous dose. In one embodiment, each subsequent dose is administered one month after the previous dose of collagen 7, or the functional fragment or variant thereof.

In one embodiment, the collagen 7, or functional fragment or variant thereof, is administered once a month for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 6 weeks, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 2 months, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered systemically, e.g., intravenously. In one embodiment, the collagen 7, or functional fragment or variant thereof, is administered topically, e.g., a cream, ointment, or lotion.

In some embodiments, the collagen 7, or functional fragment or variant thereof, is administered in combination with one or more additional agents, e.g., a chemotherapeutic agent, immunosuppressants, antibiotics, analgesics, opioids, anti-virals, or anti-inflammatory agents. In one embodiment, the collagen 7, or functional fragment or variant thereof, is administered in combination with one or more chemotherapeutic agents. In some embodiments, the chemotherapeutic agent is administered systemically, e.g., intravenously. In some embodiments, the chemotherapeutic agent is administered topically.

In one aspect, the disclosure features a method of treating a subject, the method comprising: administering to an elderly subject having a skin cancer or at risk of having a skin cancer, collagen 7 or functional fragments and variants thereof.

In certain embodiments, the method further comprises selecting a subject for administration of collagen 7, or functional fragments or variants thereof, based at least in part upon the determination that the subject is an elderly subject. In certain embodiments, the method comprises selecting a subject for administration of collagen 7, or functional fragments or variants thereof, based, at least in part, upon the determination that the subject is elderly and has at least one risk factor for skin cancer. In certain embodiments, the method comprises selecting a subject for administration of collagen 7, or functional fragments or variants thereof, based, at least in part, upon the determination that the subject is elderly and the subject has skin cancer.

In certain embodiments, the elderly subject is a subject at least 60 or over, e.g., over the age of 65, over the age of 70, over the age of 75, over the age of 80, over the age of 85, over the age of 90, over the age of 95, over the age of 100, over the age of 105, over the age of 110, or over the age of 115.

In certain embodiments, the elderly subject is at least 60 years of age, at least 65 years of age, at least 70 years of age, at least 75 years of age, at least 80 years of age, at least 85 years of age, at least 90 years of age, at least 95 years of age, at least 100 years of age, at least 105 years of age, at least 110 years of age, or at least 115 years of age.

In certain embodiments, the elderly subject is between the ages of 60-120, between the ages of 60-110, between the ages of 60-100, between the ages of 60-90, between the ages of 60-80.

In certain embodiments, the elderly subject is between the ages of 60-70, between the ages of 70-80, between the ages of 80-90, between the ages of 90-100, between the ages of 100-110, or between the ages of 110-120.

In certain embodiments, the elderly subject is between the ages of 60-65, between the ages of 65-70, between the ages of 70-75, between the ages of 75-80, between the ages of 80-85, between the ages of 85-90, between the ages of 90-95, between the ages of 95-100, between the ages of 100-105, or between the ages of 105-110, between the ages of 110-115.

In some embodiments, the elderly subject has one or more risk factor for developing a skin cancer. In some embodiments, the elderly subject has one or more, two or more, three or more, four or more, five or more, or six or more risk factors for developing a skin cancer. In some embodiments, the elderly subject has one or more, two or more, three or more, four or more, five or more, or six or more of the following risk factors for developing a skin cancer: a previous diagnosis of a skin cancer, e.g., squamous cell carcinoma; melanoma, e.g., superficial spreading melanoma, nodular melanoma, lentigo maligna melanoma, acral lentiginous melanoma; basal cell carcinoma; or Kaposi's sarcoma; previous recipient of an organ transplant, e.g., a solid organ transplant recipient; previous or current use of a tobacco product, e.g., cigarette smoking, cigar smoking, chewing tobacco, dipping or spit tobacco; long term sun exposure, long term daily sun exposure; or a skin type associated with an risk of susceptibility to skin cancer, e.g., skin phototype I, skin phototype II, skin phototype III.

In some embodiments, the skin cancer is a squamous cell carcinoma; melanoma, e.g., superficial spreading melanoma, nodular melanoma, lentigo maligna melanoma, acral lentiginous melanoma; basal cell carcinoma; or Kaposi's sarcoma. In some embodiments, the skin cancer is squamous cell carcinoma. In some embodiments, the skin cancer is associated with the oral mucosa, e.g., lip, tongue, oral cavity, or oropharynx. In some embodiments, the skin cancer is associated with sun protected skin, e.g., parts of the body generally protected from sun exposure, e.g. trunk. In some embodiments, the cancer is associated with non-sun protected skin, e.g., parts of the body generally exposed to the sun, e.g., face, ears, neck, extremities, e.g., back of the hands, arms, legs.

In some embodiments, the collagen 7, or the functional fragment or variant thereof, is administered as a single dose. In some embodiments, the collagen 7, or the functional fragment or variant thereof, is administered as multiple doses (an initial dose and one or more subsequent doses) over a period of time.

In one embodiment, the multiple doses of the collagen 7, or the functional fragment or variant thereof, are administered for a period of at least 3 months, 4 months, 5 months, 6 months, 9 months, 12 months, 15 months, 18 months, 21 months, 2 years, three years, four years, five years, six years or more (e.g., over a lifetime).

In one embodiment, each subsequent dose of collagen 7, or the functional fragment or variant thereof, is administered two weeks, three weeks, four weeks, five weeks, six weeks, eight weeks after the previous dose. In one embodiment, each subsequent dose is administered one month after the previous dose of collagen 7, or the functional fragment or variant thereof.

In one embodiment, the collagen 7, or functional fragment or variant thereof, is administered once a month for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 6 weeks, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 2 months, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered systemically, e.g., intravenously. In one embodiment, the collagen 7, or functional fragment or variant thereof, is administered topically, e.g., a cream applied to the site of a skin cancer lesion.

In some embodiments, the collagen 7, or functional fragment or variant thereof, is administered in combination with one or more additional agents, e.g., a chemotherapeutic agent, an immunotherapeutic agent. In one embodiment, the collagen 7, or functional fragment or variant thereof, is administered in combination with one or more chemotherapeutic agents. In some embodiments, the chemotherapeutic agent is administered systemically, e.g., intravenously. In some embodiments, the chemotherapeutic agent is administered topically, e.g., a cream, liquid, or ointment applied to the site of a skin cancer lesion. In one embodiment, the chemotherapeutic agent is 5-flurouracil. In one embodiment, the chemotherapeutic agent is 5-flurouracil for the treatment of premalignant actinic keratosis or squamous cell carcinomas. In some embodiments, the chemotherapeutic agent is imiquimod. In some embodiments, the chemotherapeutic agent is imiquimod for the treatment of the treatment of premalignant actinic keratosis or squamous cell carcinomas. In some embodiments the chemotherapeutic agent is an inhibitor of the hedgehog signaling pathway, e.g., vismodegib. In some embodiments the chemotherapeutic agent is an inhibitor of the hedgehog signaling pathway, e.g., vismodegib, for the treatment of basal cell carcinoma. In some embodiments the chemotherapeutic agent is an inhibitor of the hedgehog signaling pathway, e.g., vismodegib, for the treatment of melanoma. In some embodiments the chemotherapeutic agent is an inhibitor of the hedgehog signaling pathway, e.g., vismodegib, for the treatment of metastatic melanoma. In some embodiments, the chemotherapeutic agent is dacarbazine. In some embodiments, the chemotherapeutic agent is dacarbazine in combination with carmustin and/or tamoxifen. In some embodiments, the chemotherapeutic agent is dacarbazine in combination with cisplatin and/or vinblastine. In some embodiments, the chemotherapeutic agent is temozolomide. In one embodiment, the collagen 7, or functional fragment or variant thereof, is administered in combination with one or more immunotherapeutic and/or chemotherapeutic agents. In one embodiment, the immunotherapeutic agent is an interferon, e.g., interferon alpha-2b. In one embodiment, the immunotherapeutic agent is tumor necrosis factor. In one embodiment, the immunotherapeutic agent is a lymphokine, e.g., IL-2. In one embodiment, the immunotherapeutic agent is tumor infilitrating lymphokines with or without IL-2. In one embodiment, the immunotherapeutic agent is an anti-CTLA-4 agent, e.g., and anti-CTLA4 antibody, e.g., ipilimumab (MDX-010, MDX-101). In one embodiment, the immunotherapeutic agent is a gene expression inhibiting compound, e.g., PLX4032.

In one aspect, the disclosure features a method of treating a subject, the method comprising: administering to an elderly subject having vaginal tearing, e.g., tearing, abrasion, or erosion of the skin around the vagina, e.g., vaginal opening, vaginal tissue; or at risk of having vaginal tearing, collagen 7 or functional fragments and variants thereof.

In certain embodiments, the method further comprises selecting a subject for administration of collagen 7, or functional fragments or variants thereof, based at least in part upon the determination that the subject is an elderly subject. In certain embodiments, the method comprises selecting a subject for administration of collagen 7, or functional fragments or variants thereof, based, at least in part, upon the determination that the subject is elderly and has a vaginal tear or at risk of having vaginal tearing.

In certain embodiments, the elderly subject is at least 60 or over, e.g., over the age of 65, over the age of 70, over the age of 75, over the age of 80, over the age of 85, over the age of 90, over the age of 95, over the age of 100, over the age of 105, over the age of 110, or over the age of 115.

In certain embodiments, the elderly subject is at least 60 years of age, at least 65 years of age, at least 70 years of age, at least 75 years of age, at least 80 years of age, at least 85 years of age, at least 90 years of age, at least 95 years of age, at least 100 years of age, at least 105 years of age, at least 110 years of age, or at least 115 years of age.

In certain embodiments, the elderly subject is between the ages of 60-120, between the ages of 60-110, between the ages of 60-100, between the ages of 60-90, between the ages of 60-80.

In certain embodiments, the elderly subject is between the ages of 60-70, between the ages of 70-80, between the ages of 80-90, between the ages of 90-100, between the ages of 100-110, or between the ages of 110-120.

In certain embodiments, the elderly subject is between the ages of 60-65, between the ages of 65-70, between the ages of 70-75, between the ages of 75-80, between the ages of 80-85, between the ages of 85-90, between the ages of 90-95, between the ages of 95-100, between the ages of 100-105, or between the ages of 105-110, between the ages of 110-115.

In some embodiments, the elderly subject has one or more risk factors for vaginal tearing. In some embodiments, the elderly subject has one or more, two or more, three or more, four or more, five or more, or six or more risk factors for developing vaginal tearing. In some embodiments, the elderly subject has one or more, two or more, three or more, four or more, five or more, or six or more of the following risk factors for developing vaginal tearing: a level of estrogen commonly associated with menopause or post menopause, e.g., below 50 pg/ml in a blood sample, below 40 pg/ml in a blood sample, below 30 pg/ml in a blood sample, below 20 pg/ml in a blood sample, below 10 pg/ml in a blood sample; menopausal; post-menopausal; sexually active; not currently receiving estrogen therapy; a contraindication to estrogen therapy, e.g., irregular involuntary spasms of limbs or facial muscles, tobacco smoking, epileptic seizure, migraine headache, a blood clot within the blood vessels of the eye, increased cardiovascular event risk, mammography abnormal, protein c deficiency disease, hyperlipoproteinemia, high blood pressure, severe uncontrolled high blood pressure, heart attack, disease of the arteries of the heart, blood clot in lung, stroke, obstruction of a blood vessel by a blood clot, blood clot in vein, blood clot in a deep vein, asthma, liver problems, disease of the gallbladder, lump in the breast, endometriosis, bleeding not related to menstrual period, yellow-brown patches on skin, systemic lupus erythematosus, pregnancy, uterine fibroids, breast cancer, family history of breast cancer, cancer of the ovary, cancer in the lining of the uterus, tumor that is dependent on estrogen for growth, underactive thyroid, diabetes, high cholesterol, high amount of triglyceride in the blood, low amount of calcium in the blood, high amount of calcium in the blood, water retention, hepatic porphyria, inherited disorder of continuing episodes of swelling, overweight, deficiency of anti-clotting agents, abnormal increase in ability of blood to clot, disorder of mental processes due to a brain disease; cancer, e.g., breast cancer, cancer of the ovary, cancer in the lining of the uterus, a cancer dependent on estrogen for growth, a cancer in remission, e.g., breast cancer, cancer of the ovary, cancer in the lining of the uterus, a cancer dependent on estrogen for growth; an estrogen allergy.

In some embodiments, the collagen 7, or the functional fragment or variant thereof, is administered as a single dose. In some embodiments, the collagen 7, or the functional fragment or variant thereof, is administered as multiple doses (an initial dose and one or more subsequent doses) over a period of time.

In one embodiment, the collagen 7, or the functional fragment or variant thereof, is administered for a period of at least 3 months, 4 months, 5 months, 6 months, 9 months, 12 months, 15 months, 18 months, 21 months, 2 years, three years, four years, five years, six years or more (e.g., over a lifetime).

In one embodiment, each subsequent dose of collagen 7, or the functional fragment or variant thereof, is administered two weeks, three weeks, four weeks, five weeks, six weeks, eight weeks after the previous dose. In one embodiment, each subsequent dose is administered one month after the previous dose of collagen 7, or the functional fragment or variant thereof.

In one embodiment, the collagen 7, or functional fragment or variant thereof, is administered once a month for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 6 weeks, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 2 months, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered systemically, e.g., intravenously. In one embodiment, the collagen 7, or functional fragment or variant thereof, is administered topically.

In some embodiments, the collagen 7, or functional fragment or variant thereof, is administered in combination with one or more additional agents. In some embodiments, the additional agent is a vaginal moisturizer, e.g., a non-estrogen based vaginal moisturizer. In some embodiments, the additional agent is an antidepressant, e.g., a low dose antidepressant, e.g., vanlefaxine (effexor); a selective serotonin uptake inhibitor, e.g., fluoxetine (prozax, sarafem), paroxetine (paxil), citalopram (celexa), sertraline (Zoloft). In one embodiment the additional agent is an agent to which reduces the frewuency or severity of hot flashes, e.g., gabapentin (neurotonin), clonidine. In some embodiments, the additional agent is an anti-osteoporosis agent, e.g., a bisphosphate, e.g., alendronate (Fosamax), risedronate (Actonel) and ibandronate (Boniva).

In one aspect, the disclosure features a method of treating a subject, the method comprising: administering to an elderly or chronically ill subject who has had a skin lesion, e.g., a skin cancer, that has been surgically excised, collagen 7 or functional fragments and variants thereof.

In certain embodiments, the method further comprises selecting a subject for administration of collagen 7, or functional fragments or variants thereof, based at least in part upon the determination that the subject is an elderly subject or a chronically ill subject. In certain embodiments, the method comprises selecting a subject for administration of collagen 7, or functional fragments or variants thereof, based, at least in part, upon the determination that the subject is elderly and/or chronically ill and has a skin lesion. The method can further comprise removing the skin lesion.

In certain embodiments, the elderly subject is at least 60 or over, e.g., over the age of 65, over the age of 70, over the age of 75, over the age of 80, over the age of 85, over the age of 90, over the age of 95, over the age of 100, over the age of 105, over the age of 110, or over the age of 115.

In certain embodiments, the elderly subject is at least 60 years of age, at least 65 years of age, at least 70 years of age, at least 75 years of age, at least 80 years of age, at least 85 years of age, at least 90 years of age, at least 95 years of age, at least 100 years of age, at least 105 years of age, at least 110 years of age, or at least 115 years of age.

In certain embodiments, the elderly subject is between the ages of 60-120, between the ages of 60-110, between the ages of 60-100, between the ages of 60-90, between the ages of 60-80.

In certain embodiments, the elderly subject is between the ages of 60-70, between the ages of 70-80, between the ages of 80-90, between the ages of 90-100, between the ages of 100-110, or between the ages of 110-120.

In certain embodiments, the elderly subject is between the ages of 60-65, between the ages of 65-70, between the ages of 70-75, between the ages of 75-80, between the ages of 80-85, between the ages of 85-90, between the ages of 90-95, between the ages of 95-100, between the ages of 100-105, or between the ages of 105-110, between the ages of 110-115.

In some embodiments, the skin lesion is unable to be or is not recommended to be surgically closed after excision, e.g., a burn, an infected skin lesion, e.g., bacterially infected skin lesion. In some embodiments, the excision is followed by secondary wound closure (i.e., open healing, closure by secondary intention), e.g., the skin edges of the excision are not sutured together, but left open.

In some embodiments, the skin lesion is a benign skin lesion. In some embodiments, the skin lesion is a malignant skin lesion. In some embodiments, the skin lesion is a skin cancer, e.g., squamous cell carcinoma; melanoma, e.g., superficial spreading melanoma, nodular melanoma, lentigo maligna melanoma, acral lentiginous melanoma; basal cell carcinoma or Kaposi's sarcoma. In some embodiments, the skin lesion is a burn, an infected skin lesion, e.g., bacterially infected skin lesion.

In some embodiments, the collagen 7, or the functional fragment or variant thereof, is administered as a single dose. In some embodiments, the collagen 7, or the functional fragment or variant thereof, is administered as multiple doses (an initial dose and one or more subsequent doses) over a period of time.

In one embodiment, multiple doses of the collagen 7, or the functional fragment or variant thereof, are administered for a period of at least 3 months, 4 months, 5 months, 6 months, 9 months, 12 months, 15 months, 18 months, 21 months, 2 years, three years, four years, five years, six years or more (e.g., over a lifetime).

In one embodiment, each subsequent dose of collagen 7, or the functional fragment or variant thereof, is administered two weeks, three weeks, four weeks, five weeks, six weeks, eight weeks after the previous dose. In one embodiment, each subsequent dose is administered one month after the previous dose of collagen 7, or the functional fragment or variant thereof.

In one embodiment, the collagen 7, or functional fragment or variant thereof, is administered once a month for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 6 weeks, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 2 months, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered systemically, e.g., intravenously. In one embodiment, the collagen 7, or functional fragment or variant thereof, is administered topically.

In some embodiments, the collagen 7, or functional fragment or variant thereof, is administered in combination with one or more additional agents, e.g., chemotherapeutic agents, immunosuppressants, antibiotics, analgesics, opioids, anti-virals, or anti-inflammatory agents.

In one aspect, the disclosure features a method of treating a subject at risk of having cancer, e.g., skin cancer, the method comprising: selecting a subject that has received an organ transplant, and administering collagen 7 or functional fragments and variants thereof.

In some embodiments, the organ transplant is a solid organ transplant, e.g., heart, liver, kidney, lung, pancreas, intestine, stomach, testis etc. as contrasted to liquid transplanted tissues, e.g., bone marrow, pancreatic islets, etc.

In some embodiments the subject is elderly. In certain embodiments, the elderly subject is a subject at least 60 or over, e.g., over the age of 65, over the age of 70, over the age of 75, over the age of 80, over the age of 85, over the age of 90, over the age of 95, over the age of 100, over the age of 105, over the age of 110, or over the age of 115.

In certain embodiments, the elderly subject is at least 60 years of age, at least 65 years of age, at least 70 years of age, at least 75 years of age, at least 80 years of age, at least 85 years of age, at least 90 years of age, at least 95 years of age, at least 100 years of age, at least 105 years of age, at least 110 years of age, or at least 115 years of age.

In certain embodiments, the elderly subject is between the ages of 60-120, between the ages of 60-110, between the ages of 60-100, between the ages of 60-90, between the ages of 60-80.

In certain embodiments, the elderly subject is between the ages of 60-70, between the ages of 70-80, between the ages of 80-90, between the ages of 90-100, between the ages of 100-110, or between the ages of 110-120.

In certain embodiments, the elderly subject is between the ages of 60-65, between the ages of 65-70, between the ages of 70-75, between the ages of 75-80, between the ages of 80-85, between the ages of 85-90, between the ages of 90-95, between the ages of 95-100, between the ages of 100-105, or between the ages of 105-110, between the ages of 110-115.

In certain embodiments, the subject has been diagnosed with a skin cancer, e.g., a squamous cell carcinoma; melanoma, e.g., superficial spreading melanoma, nodular melanoma, lentigo maligna melanoma, acral lentiginous melanoma; basal cell carcinoma; or Kaposi's sarcoma.

In some embodiments, the collagen 7, or the functional fragment or variant thereof, is administered as a single dose. In some embodiments, the collagen 7, or the functional fragment or variant thereof, is administered as multiple doses (an initial dose and one or more subsequent doses) over a period of time.

In one embodiment, multiple doses of the collagen 7, or the functional fragment or variant thereof, are administered for a period of at least 3 months, 4 months, 5 months, 6 months, 9 months, 12 months, 15 months, 18 months, 21 months, 2 years, three years, four years, five years, six years or more (e.g., over a lifetime).

In one embodiment, each subsequent dose of collagen 7, or the functional fragment or variant thereof, is administered two weeks, three weeks, four weeks, five weeks, six weeks, eight weeks after the previous dose. In one embodiment, each subsequent dose is administered one month after the previous dose of collagen 7, or the functional fragment or variant thereof.

In one embodiment, the collagen 7, or functional fragment or variant thereof, is administered once a month for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 6 weeks, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 2 months, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered systemically, e.g., intravenously. In one embodiment, the collagen 7, or functional fragment or variant thereof, is administered topically, e.g., a cream applied to the site of a skin cancer lesion.

In some embodiments, the collagen 7, or functional fragment or variant thereof, is administered in combination with one or more additional agents. In some embodiments the additional agent includes one or more of an immunosuppressant agent; an infection fighting agent, e.g., an antibiotic, anti-viral, anti-fungal; and or a nutritional supplement. In some embodiments, the additional agent is an immunosuppressant, e.g., steroids, cyclosporine A, azathioprine, prednisone, FK 506, mycophenolate mofetil, tacrolimus, muromonab-CD3, daclizumab. In some embodiments, the collagen 7, or functional fragment or variant thereof, is administered in combination with an immunosuppressant and an infection fighting agent, e.g., an antibiotic, anti-viral, or anti-fungal. In some embodiments, the collagen 7, or functional fragment or variant thereof, is administered in combination with an immunosuppressant and a nutritional supplement, e.g., a vitamin, iron, magnesium, calcium supplement.

In one aspect, the disclosure features a method of treating a subject, the method comprising: selecting a subject diagnosed with a disorder in which the treatment of the disorder is an organ transplant, and administering to the subject collagen 7 or functional fragments and variants thereof.

In some embodiments, the subject is diagnosed with failure of a solid organ, e.g., kidney failure, heart failure, liver failure, lung failure, pancreatic failure; or diagnosed with a disorder which would lead to failure of a solid organ, e.g., cancer of a solid organ, e.g., liver cancer, lung cancer, pancreatic cancer; kidney cancer, testicular cancer, intestinal cancer, stomach cancer, heart cancer; chronic renal failure; acute renal failure; type 1 diabetes; type 2 diabetes; chronic kidney disease; immune system conditions, e.g., lupus, chronic viral illnesses, e.g., HIV/AIDS, hepatitis B, hepatitis C; urine blockage in the kidneys; kidney damage; impaired blood flow to the kidneys; congestive heart failure; coronary artery disease; high blood pressure; faulty heart valves; cardiomyopathy; myocarditis; heart arrhythmias; acute heart failure; chronic heart failure; liver failure; liver cirrhosis; biliary duct atresia; cystic fibrosis; early stage liver cancer; primary biliary cirrhosis; primary sclerosing cholangitis; Wilson's disease; lung cancer.

In some embodiments the subject is elderly. In certain embodiments, the elderly subject is at least 60 or over, e.g., over the age of 55, over the age of 70, over the age of 75, over the age of 80, over the age of 85, over the age of 90, over the age of 95, over the age of 100, over the age of 105, over the age of 110, or over the age of 115.

In certain embodiments, the elderly subject is at least 60 years of age, at least 65 years of age, at least 70 years of age, at least 75 years of age, at least 80 years of age, at least 85 years of age, at least 90 years of age, at least 95 years of age, at least 100 years of age, at least 105 years of age, at least 110 years of age, or at least 115 years of age.

In certain embodiments, the elderly subject is between the ages of 60-120, between the ages of 60-110, between the ages of 60-100, between the ages of 60-90, between the ages of 60-80.

In certain embodiments, the elderly subject is between the ages of 60-70, between the ages of 70-80, between the ages of 80-90, between the ages of 90-100, between the ages of 100-110, or between the ages of 110-120.

In certain embodiments, the elderly subject is between the ages of 60-65, between the ages of 65-70, between the ages of 70-75, between the ages of 75-80, between the ages of 80-85, between the ages of 85-90, between the ages of 90-95, between the ages of 95-100, between the ages of 100-105, or between the ages of 105-110, between the ages of 110-115.

In certain embodiments, the subject has been diagnosed with a skin cancer, e.g., a squamous cell carcinoma; melanoma, e.g., superficial spreading melanoma, nodular melanoma, lentigo maligna melanoma, acral lentiginous melanoma; basal cell carcinoma; or Kaposi's sarcoma.

In some embodiments, the collagen 7, or the functional fragment or variant thereof, is administered as a single dose. In some embodiments, the collagen 7, or the functional fragment or variant thereof, is administered as multiple doses (an initial dose and one or more subsequent doses) over a period of time.

In one embodiment, the multiple doses of the collagen 7, or the functional fragment or variant thereof, are administered for a period of at least 3 months, 4 months, 5 months, 6 months, 9 months, 12 months, 15 months, 18 months, 21 months, 2 years, three years, four years, five years, six years or more (e.g., over a lifetime).

In one embodiment, each subsequent dose of collagen 7, or the functional fragment or variant thereof, is administered two weeks, three weeks, four weeks, five weeks, six weeks, eight weeks after the previous dose. In one embodiment, each subsequent dose is administered one month after the previous dose of collagen 7, or the functional fragment or variant thereof.

In one embodiment, the collagen 7, or functional fragment or variant thereof, is administered once a month for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 6 weeks, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 2 months, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered systemically, e.g., intravenously. In one embodiment, the collagen 7, or functional fragment or variant thereof, is administered topically, e.g., a cream applied to the site of a skin cancer lesion.

In some embodiments, the collagen 7, or functional fragment or variant thereof, is administered in combination with one or more additional agents. In some embodiments the additional agent includes one or more of an immunosuppressant agent; an infection fighting agent, e.g., an antibiotic, anti-viral, anti-fungal; and or a nutritional supplement. In some embodiments, the additional agent is an immunosuppressant, e.g., steroids, cyclosporine A, azathioprine, prednisone, FK 506, mycophenolate mofetil, tacrolimus, muromonab-CD3, daclizumab. In some embodiments, the collagen 7, or functional fragment or variant thereof, is administered in combination with an immunosuppressant and an infection fighting agent, e.g., an antibiotic, anti-viral, or anti-fungal. In some embodiments, the collagen 7, or functional fragment or variant thereof, is administered in combination with an immunosuppressant and a nutritional supplement, e.g., a vitamin, iron, magnesium, calcium supplement.

### DETAILED DESCRIPTION OF THE INVENTION

Certain terms are first defined. Additional terms are defined throughout the specification.

A “chronic” or “non-healing” wound as used herein, refers to a wound that does not heal in an amount of time considered predictable by those skilled in the art for the characteristics of the wound. Chronic wounds are those that do not progress through the usual phases of healing. Examples of common chronic wounds can include, pressure sores, chronic ulcers, bed sores, burn wounds, lower extremity ulcers, lower extremity venous ulcers, lower extremity stasis ulcers, surgical wounds, diabetic ulcers, arterial wounds, and radiation ulcers.

A “chronically ill” subject as used herein, refers to a subject diagnosed with a chronic disorder.

A “chronic disorder”, as used herein, refers to a disorder of long duration and, often, of generally slow progression. Such disorder include those that require continued therapy, e.g., for at least one year, 2 years, 3 years, 4 years, 5 years, 6 years, 7 years, 8 years, 9 years, 10 years, or for the rest of the patient's life. Exemplary chronic disorders can include diabetes, cardiovascular disease, chronic respiratory diseases, lupus, multiple sclerosis, AIDS.

“Chronic administration”, as used herein, refers to the administration of more than one dose of an agent over a period of time. Chronic administration can include regular administration for an extended period of time. Chronic administration can also include the administration of therapy over a prolonged period of time (in some cases, for the duration of a subject's lifetime) so that the concentration of the therapeutic agent is maintained at a therapeutically or prophylactically effective level throughout the course of treatment.

An “effective amount” of collagen 7 or functional fragment or variant thereof refers to the amount of collagen 7 or functional fragment or variant thereof, when administered in an aggregate of multiple doses, or as part of any other type of defined treatment regimen, produces a measureable statistical improvement in outcome, as evidenced by at least one clinical parameter associated with the complication.

“Recombinant”, as used herein, in reference to a protein or polypeptide molecule, pertains to a protein or polypeptide molecule expressed utilizing isolated nucleic acid molecules or recombinant nucleic acid molecules.

A skin type associated with a higher than average risk of developing skin cancer can include skin phototypes I, II, and II as described herein. Skin type can be classified into six skin phototypes. Phototypes I and II are associated with the highest risk of skin cancer. Phototype I is described as skin which always burns and does not tan when exposed to the sun, e.g., exposed to 30 minutes of sun light. Phototype II is described as skin which almost always or usually burns and rarely tans when exposed to the sun. Phototype III is described as skin which sometimes burns and sometimes tans when exposed to the sun. Phototype IV is described as skin which tends to tan easily and is less likely to burn when exposed to sun. Phototype V and VI are described as skin which tans easily and rarely burns when exposed to the sun, e.g., Hispanic skin, Black skin, e.g., African American skin, aboriginal skin.

“Collagen 7” as used herein refers to collagen type 7 encoded by the COL7A1 gene. Collagen 7 consists of 2,944 amino acids. It comprises a non-collagenous NC1 domain (residues 1-1253), the central collagenous helical domain (residues 1254-2783), and the carboxyl-terminal NC2 domain (residues 2784-2944).

A functional fragment of collagen 7 refers to a portion of collagen 7 that maintains the ability to form anchoring fibrils between the epidermal and dermal layers of human skin, and the ability to bind collagen 4 and laminin-332. For example, a functional fragment can include all or a portion of the NC1 domain and/or the NC2 domain of collagen 7, e.g., the functional fragment can be collagen 7 without all of a portion of the central collagenous helical domain, e.g., a fragment that does not include amino acid residues 1920-2603 of the central collagenous helical domain of collagen 7.

A variant of collagen 7 refers to a polypeptide that has substantial identity with collagen 7 that maintains the ability to form anchoring fibrils between the epidermal and dermal layers of human skin. Collagen 7 variants include, but are not limited to, collagen 7 polypeptides that have been either chemically modified relative to collagen 7 and/or contain one or more amino acid sequence alterations relative to collagen 7.

Variants of collagen 7 include polypeptides having at least 75%, 80%, 85%, 90%, 95%, 96%, 97%, 98% or 99% identity with the amino acid sequence of human collagen 7 (SEQ ID NO:2). Calculations of “identity” or “sequence homology” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

Variants of collagen 7 also include polypeptides having amino acid modifications (e.g., deletions, additions or substitutions, such as conservative substations) from the amino acid sequence of collagen 7 (SEQ ID NO:2). For example, a variant of collagen 7 can differ by at least 1, 2, 3, 4, 5 but not more than 50, 40, 30, 20, 15 or 10 amino acids from collagen 7 (SEQ ID NO:2). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Collagen 7 composition” refers to a plurality of collagen 7 polypeptides or collagen 7 equivalent polypeptides, or functional fragments or variants of collagen 7, including variants and chemically modified forms that have been separated from the cell in which they were synthesized. An “isolated composition” refers to a composition that is removed from at least 90% of at least one component of a natural sample from which the isolated composition can be obtained. Compositions produced artificially or naturally can be “compositions of at least” a certain degree of purity if the species or population of species of interests is at least 5, 10, 25, 50, 75, 80, 90, 92, 95, 98, or 99% pure on a weight-weight basis.

An “isolated” protein refers to a protein that is removed from at least 90% of at least one component of a natural sample from which the isolated protein can be obtained. Proteins can be “of at least” a certain degree of purity if the species or population of species of interest is at least 5, 10, 25, 50, 75, 80, 90, 92, 95, 98, or 99% pure on a weight-weight basis.

The term “preventing” a disease in a subject refers to subjecting the subject to a pharmaceutical treatment, e.g., the administration of a drug, such that at least one symptom of the disease is prevented, that is, administered prior to clinical manifestation of the unwanted condition (e.g., disease or other unwanted state of the host animal) so that it protects the host against developing the unwanted condition. “Preventing” a disease may also be referred to as “prophylaxis” or “prophylactic treatment.”

“Treating” or “treating” a subject having a disorder, refers to subjecting the subject to a pharmaceutical treatment, e.g., the administration of a drug, such that at least one symptom of the disease is cured, alleviated or decreased.

A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein to elicit a desired response in the individual. A therapeutically effective amount is also one in which any toxic or detrimental effects of the composition are outweighed by the therapeutically beneficial effects.

A “patient”, “subject” or “host” (these terms are used interchangeably) to be treated by the subject method may mean either a human or non-human animal.

Any of the treatments described herein can be administered in combination with another agent or therapy. The term “combination” refers to the use of the two or more agents or therapies to treat the same patient, wherein the use or action of the agents or therapies overlap in time. The agents or therapies can be administered at the same time (e.g., as a single formulation that is administered to a patient or as two separate formulations administered concurrently) or sequentially in any order.

### Collagen 7

As a major component of anchoring fibrils, collagen 7 functions in maintaining tissue integrity. Anchoring fibrils are structural elements that serve as attachment complexes at the interface between the epithelial and mesenchymal layers of several tissues, including the skin, oral mucosa, and cervix (Chung et al. Dermatol Clin 28(1): 93-105 (2010)). In the skin, anchoring fibrils extend from the lower portion of the epidermal basement membrane to the underlying papillary dermis, securing the association between the epidermal basement membrane and the papillary dermis (Varki et al. J Med Genet 44:181-192 (2007)). This association aids to provide and maintain cohesion between the epidermis and dermis, contributing to the integrity to the skin, which is critical for its proper structure, function, and homeostasis (Villone et al. J Blot Chem 283(36): 24506-24513 (2008)). The Collagen 7 nucleotide and amino acid sequences are known in the art. An exemplary nucleotide and amino acid sequence for human Collagen 7 is provided herein as SEQ ID NO:1 and SEQ ID NO:2, respectively.

| NCBI Reference Sequence: NM_000094 | |
|---|---|
| (SEQ ID NO: 1) | |
| 1 | gatgacgctg cggcttctgg tggccgcgct ctgcgccggg atcctggcag aggcgccccg |
| 61 | agtgcgagcc cagcacaggg agagagtgac ctgcacgcgc ctttacgccg ctgacattgt |
| 121 | gttcttactg gatggctcct catccattgg ccgcagcaat ttccgcgagg tccgcagctt |
| 181 | tctcgaaggg ctggtgctgc ctttctctgg agcagccagt gcacagggtg tgcgctttgc |
| 241 | cacagtgcag tacagcgatg acccacggac agagttcggc ctggatgcac ttggctctgg |
| 301 | gggtgatgtg atccgcgcca tccgtgagct tagctacaag gggggcaaca ctcgcacagg |
| 361 | ggctgcaatt ctccatgtgg ctgaccatgt cttcctgccc cagctggccc gacctggtgt |
| 421 | ccccaaggtc tgcatcctga tcacagacgg gaagtcccag gacctggtgg acacagctgc |
| 481 | ccaaaggctg aaggggcagg gggtcaagct atttgctgtg gggatcaaga atgctgaccc |
| 541 | tgaggagctg aagcgagttg cctcacagcc caccagtgac ttcttcttct tcgtcaatga |
| 601 | cttcagcatc ttgaggacac tactgcccct cgtttcccgg agagtgtgca cgactgctgg |
| 661 | tggcgtgcct gtgacccgac ctccggatga ctcgacctct gctccacgag acctggtgct |
| 721 | gtctgagcca agcagccaat ccttgagagt acagtggaca gcggccagtg gccctgtgac |
| 781 | tggctacaag gtccagtaca ctcctctgac ggggctggga cagccactgc cgagtgagcg |
| 841 | gcaggaggtg aacgtcccag ctggtgagac cagtgtgcgg ctgcggggtc tccggccact |
| 901 | gaccgagtac caagtgactg tgattgccct ctacgccaac agcatcgggg aggctgtgag |
| 961 | cgggacagct cggaccactg ccctagaagg gccggaactg accatccaga ataccacagc |
| 1021 | ccacagcctc ctggtggcct ggcggagtgt gccaggtgcc actggctacc gtgtgacatg |
| 1081 | gcgggtcctc agtggtgggc ccacacagca gcaggagctg ggccctgggc agggttcagt |
| 1141 | gttgctgcgt gacttggagc ctggcacgga ctatgaggtg accgtgagca ccctatttgg |
| 1201 | ccgcagtgtg gggcccgcca cttccctgat ggctcgcact gacgcttctg ttgagcagac |
| 1261 | cctgcgcccg gtcatcctgg gccccacatc catcctcctt tcctggaact tggtgcctga |
| 1321 | ggcccgtggc taccggttgg aatggcggcg tgagactggc ttggagccac cgcagaaggt |
| 1381 | ggtactgccc tctgatgtga cccgctacca gttggatggg ctgcagccgg gcactgagta |
| 1441 | ccgcctcaca ctctacactc tgctggaggg ccacgaggtg gccacccctg caaccgtggt |
| 1501 | tcccactgga ccagagctgc ctgtgagccc tgtaacagac ctgcaagcca ccgagctgcc |
| 1561 | cgggcagcgg gtgcgagtgt cctggagccc agtccctggt gccacccagt accgcatcat |
| 1621 | tgtgcgcagc acccaggggg ttgagcggac cctggtgctt cctgggagtc agacagcatt |
| 1681 | cgacttggat gacgttcagg ctgggcttag ctacactgtg cgggtgtctg ctcgagtggg |
| 1741 | tccccgtgag ggcagtgcca gtgtcctcac tgtccgccgg gagccggaaa ctccacttgc |
| 1801 | tgttccaggg ctgcgggttg tggtgtcaga tgcaacgcga gtgagggtgg cctggggacc |
| 1861 | cgtccctgga gccagtggat ttcggattag ctggagcaca ggcagtggtc cggagtccag |
| 1921 | ccagacactg cccccagact ctactgccac agacatcaca gggctgcagc ctggaaccac |
| 1981 | ctaccaggtg gctgtgtcgg tactgcgagg cagagaggag ggccctgctg cagtcatcgt |
| 2041 | ggctcgaacg gacccactgg gcccagtgag gacggtccat gtgactcagg ccagcagctc |
| 2101 | atctgtcacc attacctgga ccagggttcc tggcgccaca ggatacaggg tttcctggca |
| 2161 | ctcagcccac ggcccagaga aatcccagtt ggtttctggg gaggccacgg tggctgagct |
| 2221 | ggatggactg gagccagata ctgagtatac ggtgcatgtg agggcccatg tggctggcgt |
| 2281 | ggatgggccc cctgcctctg tggttgtgag gactgcccct gagcctgtgg gtcgtgtgtc |
| 2341 | gaggctgcag atcctcaatg cttccagcga cgttctacgg atcacctggg taggggtcac |
| 2401 | tggagccaca gcttacagac tggcctgggg ccggagtgaa ggcggcccca tgaggcacca |
| 2461 | gatactccca ggaaacacag actctgcaga gatccggggt ctcgaaggtg gagtcagcta |
| 2521 | ctcagtgcga gtgactgcac ttgtcgggga ccgcgagggc acacctgtct ccattgttgt |
| 2581 | cactacgccg cctgaggctc cgccagccct ggggacgctt cacgtggtgc agcgcgggga |
| 2641 | gcactcgctg aggctgcgct gggagccggt gcccagagcg cagggcttcc ttctgcactg |
| 2701 | gcaacctgag ggtggccagg aacagtcccg ggtcctgggg cccgagctca gcagctatca |
| 2761 | cctggacggg ctggagccag cgacacagta ccgcgtgagg ctgagtgtcc tagggccagc |
| 2821 | tggagaaggg ccctctgcag aggtgactgc gcgcactgag tcacctcgtg ttccaagcat |
| 2881 | tgaactacgt gtggtggaca cctcgatcga ctcggtgact ttggcctgga ctccagtgtc |
| 2941 | cagggcatcc agctacatcc tatcctggcg gccactcaga ggccctggcc aggaagtgcc |
| 3001 | tgggtccccg cagacacttc cagggatctc aagctcccag cgggtgacag ggctagagcc |
| 3061 | tggcgtctct tacatcttct ccctgacgcc tgtcctggat ggtgtgcggg gtcctgaggc |
| 3121 | atctgtcaca cagacgccag tgtgcccccg tggcctggcg gatgtggtgt tcctaccaca |
| 3181 | tgccactcaa gacaatgctc accgtgcgga ggctacgagg agggtcctgg agcgtctggt |
| 3241 | gttggcactt gggcctcttg ggccacaggc agttcaggtt ggcctgctgt cttacagtca |
| 3301 | tcggccctcc ccactgttcc cactgaatgg ctcccatgac cttggcatta tcttgcaaag |
| 3361 | gatccgtgac atgccctaca tggacccaag tgggaacaac ctgggcacag ccgtggtcac |
| 3421 | agctcacaga tacatgttgg caccagatgc tcctgggcgc cgccagcacg taccaggggt |
| 3481 | gatggttctg ctagtggatg aacccttgag aggtgacata ttcagcccca tccgtgaggc |
| 3541 | ccaggcttct gggcttaatg tggtgatgtt gggaatggct ggagcggacc cagagcagct |
| 3601 | gcgtcgcttg gcgccgggta tggactctgt ccagaccttc ttcgccgtgg atgatgggcc |
| 3661 | aagcctggac caggcagtca gtggtctggc cacagccctg tgtcaggcat ccttcactac |
| 3721 | tcagccccgg ccagagccct gcccagtgta ttgtccaaag ggccagaagg gggaacctgg |
| 3781 | agagatgggc ctgagaggac aagttgggcc tcctggcgac cctggcctcc cgggcaggac |
| 3841 | cggtgctccc ggcccccagg ggccccctgg aagtgccact gccaagggcg agaggggctt |
| 3901 | ccctggagca gatgggcgtc caggcagccc tggccgcgcc gggaatcctg ggacccctgg |
| 3961 | agcccctggc ctaaagggct ctccagggtt gcctggccct cgtggggacc cgggagagcg |
| 4021 | aggacctcga ggcccaaagg gggagccggg ggctcccgga caagtcatcg gaggtgaagg |
| 4081 | acctgggctt cctgggcgga aaggggaccc tggaccatcg ggcccccctg gacctcgtgg |
| 4141 | accactgggg gacccaggac cccgtggccc cccagggctt cctggaacag ccatgaaggg |
| 4201 | tgacaaaggc gatcgtgggg agcggggtcc ccctggacca ggtgaaggtg gcattgctcc |
| 4261 | tggggagcct gggctgccgg gtcttcccgg aagccctgga ccccaaggcc ccgttggccc |
| 4321 | ccctggaaag aaaggagaaa aaggtgactc tgaggatgga gctccaggcc tcccaggaca |
| 4381 | acctgggtct ccgggtgagc agggcccacg gggacctcct ggagctattg gccccaaagg |
| 4441 | tgaccggggc tttccagggc ccctgggtga ggctggagag aagggcgaac gtggaccccc |
| 4501 | aggcccagcg ggatcccggg ggctgccagg ggttgctgga cgtcctggag ccaagggtcc |
| 4561 | tgaagggcca ccaggaccca ctggccgcca aggagagaag ggggagcctg gtcgccctgg |
| 4621 | ggaccctgca gtggtgggac ctgctgttgc tggacccaaa ggagaaaagg gagatgtggg |
| 4681 | gcccgctggg cccagaggag ctaccggagt ccaaggggaa cggggcccac ccggcttggt |
| 4741 | tcttcctgga gaccctggcc ccaagggaga ccctggagac cggggtccca ttggccttac |
| 4801 | tggcagagca ggacccccag gtgactcagg gcctcctgga gagaagggag accctgggcg |
| 4861 | gcctggcccc ccaggacctg ttggcccccg aggacgagat ggtgaagttg gagagaaagg |
| 4921 | tgacgagggt cctccgggtg acccgggttt gcctggaaaa gcaggcgagc gtggccttcg |
| 4981 | gggggcacct ggagttcggg ggcctgtggg tgaaaaggga gaccagggag atcctggaga |
| 5041 | ggatggacga aatggcagcc ctggatcatc tggacccaag ggtgaccgtg gggagccggg |
| 5101 | tcccccagga cccccgggac ggctggtaga cacaggacct ggagccagag agaagggaga |
| 5161 | gcctggggac cgcggacaag agggtcctcg agggcccaag ggtgatcctg gcctccctgg |
| 5221 | agcccctggg gaaaggggca ttgaagggtt tcggggaccc ccaggcccac agggggaccc |
| 5281 | aggtgtccga ggcccagcag gagaaaaggg tgaccggggt ccccctgggc tggatggccg |
| 5341 | gagcggactg gatgggaaac caggagccgc tgggccctct gggccgaatg gtgctgcagg |
| 5401 | caaagctggg gacccaggga gagacgggct tccaggcctc cgtggagaac agggcctccc |
| 5461 | tggcccctct ggtccccctg gattaccggg aaagccaggc gaggatggca aacctggcct |
| 5521 | gaatggaaaa aacggagaac ctggggaccc tggagaagac gggaggaagg gagagaaagg |
| 5581 | agattcaggc gcctctggga gagaaggtcg tgatggcccc aagggtgagc gtggagctcc |
| 5641 | tggtatcctt ggaccccagg ggcctccagg cctcccaggg ccagtgggcc ctcctggcca |
| 5701 | gggttttcct ggtgtcccag gaggcacggg ccccaagggt gaccgtgggg agactggatc |
| 5761 | caaaggggag cagggcctcc ctggagagcg tggcctgcga ggagagcctg gaagtgtgcc |
| 5821 | gaatgtggat cggttgctgg aaactgctgg catcaaggca tctgccctgc gggagatcgt |
| 5881 | ggagacctgg gatgagagct ctggtagctt cctgcctgtg cccgaacggc gtcgaggccc |
| 5941 | caagggggac tcaggcgaac agggcccccc aggcaaggag ggccccatcg gctttcctgg |
| 6001 | agaacgcggg ctgaagggcg accgtggaga ccctggccct caggggccac ctggtctggc |
| 6061 | ccttggggag aggggccccc ccgggccttc cggccttgcc ggggagcctg gaaagcctgg |
| 6121 | tattcccggg ctcccaggca gggctggggg tgtgggagag gcaggaaggc caggagagag |
| 6181 | gggagaacgg ggagagaaag gagaacgtgg agaacagggc agagatggcc ctcctggact |
| 6241 | ccctggaacc cctgggcccc ccggaccccc tggccccaag gtgtctgtgg atgagccagg |
| 6301 | tcctggactc tctggagaac agggaccccc tggactcaag ggtgctaagg gggagccggg |
| 6361 | cagcaatggt gaccaaggtc ccaaaggaga caggggtgtg ccaggcatca aaggagaccg |
| 6421 | gggagagcct ggaccgaggg gtcaggacgg caacccgggt ctaccaggag agcgtggtat |
| 6481 | ggctgggcct gaagggaagc cgggtctgca gggtccaaga ggcccccctg gcccagtggg |
| 6541 | tggtcatgga gaccctggac cacctggtgc cccgggtctt gctggccctg caggacccca |
| 6601 | aggaccttct ggcctgaagg gggagcctgg agagacagga cctccaggac ggggcctgac |
| 6661 | tggacctact ggagctgtgg gacttcctgg accccccggc ccttcaggcc ttgtgggtcc |
| 6721 | acaggggtct ccaggtttgc ctggacaagt gggggagaca gggaagccgg gagccccagg |
| 6781 | tcgagatggt gccagtggaa aagatggaga cagagggagc cctggtgtgc cagggtcacc |
| 6841 | aggtctgcct ggccctgtcg gacctaaagg agaacctggc cccacggggg cccctggaca |
| 6901 | ggctgtggtc gggctccctg gagcaaaggg agagaaggga gcccctggag gccttgctgg |
| 6961 | agacctggtg ggtgagccgg gagccaaagg tgaccgagga ctgccagggc cgcgaggcga |
| 7021 | gaagggtgaa gctggccgtg caggggagcc cggagaccct ggggaagatg gtcagaaagg |
| 7081 | ggctccagga cccaaaggtt tcaagggtga cccaggagtc ggggtcccgg gctcccctgg |
| 7141 | gcctcctggc cctccaggtg tgaagggaga tctgggcctc cctggcctgc ccggtgctcc |
| 7201 | tggtgttgtt gggttcccgg gtcagacagg ccctcgagga gagatgggtc agccaggccc |
| 7261 | tagtggagag cggggtctgg caggcccccc agggagagaa ggaatcccag gacccctggg |
| 7321 | gccacctgga ccaccggggt cagtgggacc acctggggcc tctggactca aaggagacaa |
| 7381 | gggagaccct ggagtagggc tgcctgggcc ccgaggcgag cgtggggagc caggcatccg |
| 7441 | gggtgaagat ggccgccccg gccaggaggg accccgagga ctcacggggc cccctggcag |
| 7501 | caggggagag cgtggggaga agggtgatgt tgggagtgca ggactaaagg gtgacaaggg |
| 7561 | agactcagct gtgatcctgg ggcctccagg cccacggggt gccaaggggg acatgggtga |
| 7621 | acgagggcct cggggcttgg atggtgacaa aggacctcgg ggagacaatg gggaccctgg |
| 7681 | tgacaagggc agcaagggag agcctggtga caagggctca gccgggttgc caggactgcg |
| 7741 | tggactcctg ggaccccagg gtcaacctgg tgcagcaggg atccctggtg acccgggatc |
| 7801 | cccaggaaag gatggagtgc ctggtatccg aggagaaaaa ggagatgttg gcttcatggg |
| 7861 | tccccggggc ctcaagggtg aacggggagt gaagggagcc tgtggccttg atggagagaa |
| 7921 | gggagacaag ggagaagctg gtcccccagg ccgccccggg ctggcaggac acaaaggaga |
| 7981 | gatgggggag cctggtgtgc cgggccagtc gggggcccct ggcaaggagg gcctgatcgg |
| 8041 | tcccaagggt gaccgaggct ttgacgggca gccaggcccc aagggtgacc agggcgagaa |
| 8101 | aggggagcgg ggaaccccag gaattggggg cttcccaggc cccagtggaa atgatggctc |
| 8161 | tgctggtccc ccagggccac ctggcagtgt tggtcccaga ggccccgaag gacttcaggg |
| 8221 | ccagaagggt gagcgaggtc cccccggaga gagagtggtg ggggctcctg gggtccctgg |
| 8281 | agctcctggc gagagagggg agcaggggcg gccagggcct gccggtcctc gaggcgagaa |
| 8341 | gggagaagct gcactgacgg aggatgacat ccggggcttt gtgcgccaag agatgagtca |
| 8401 | gcactgtgcc tgccagggcc agttcatcgc atctggatca cgacccctcc ctagttatgc |
| 8461 | tgcagacact gccggctccc agctccatgc tgtgcctgtg ctccgcgtct ctcatgcaga |
| 8521 | ggaggaagag cgggtacccc ctgaggatga tgagtactct gaatactccg agtattctgt |
| 8581 | ggaggagtac caggaccctg aagctccttg ggatagtgat gacccctgtt ccctgccact |
| 8641 | ggatgagggc tcctgcactg cctacaccct gcgctggtac catcgggctg tgacaggcag |
| 8701 | cacagaggcc tgtcaccctt ttgtctatgg tggctgtgga gggaatgcca accgttttgg |
| 8761 | gacccgtgag gcctgcgagc gccgctgccc accccgggtg gtccagagcc aggggacagg |
| 8821 | tactgcccag gactgaggcc cagataatga gctgagattc agcatcccct ggaggagtcg |
| 8881 | gggtctcagc agaaccccac tgtccctccc cttggtgcta gaggcttgtg tgcacgtgag |
| 8941 | cgtgcgtgtg cacgtccgtt atttcagtga cttggtcccg tgggtctagc cttcccccct |
| 9001 | gtggacaaac ccccattgtg gctcctgcca ccctggcaga tgactcactg tgggggggtg |
| 9061 | gctgtgggca gtgagcggat gtgactggcg tctgacccgc cccttgaccc |
| NCBI Reference Sequence: NP_000085: | |
| (SEQ ID NO: 2) | |
| 1 | mtlrllvaal cagilaeapr vraqhrervt ctrlyaadiv flldgsssig rsnfrevrsf |
| 61 | leglvlpfsg aasaqgvrfa tvqysddprt efgldalgsg gdvirairel sykggntrtg |
| 121 | aailhvadhv flpqlarpgv pkvcilitdg ksqdlvdtaa qrlkgqgvkl favgiknadp |
| 181 | eelkrvasqp tsdffffvnd fsilrtllpl vsrrvcttag gvpvtrppdd stsaprdlvl |
| 241 | sepssgslry qwtaasgpvt gykvqytplt glgqplpser qevnvpaget svrlrglrpl |
| 301 | teyqvtvial yansigeays gtarttaleg peltiqntta hsllvawrsv pgatgyrvtw |
| 361 | rvlsggptqg gelgpgqgsv llrdlepgtd yevtvstlfg rsvgpatslm artdasveqt |
| 421 | lrpvilgpts illswnlvpe argyrlewrr etgleppqkv vlpsdvtryq ldglqpgtey |
| 481 | rltlytlleg hevatpatvv ptgpelpvsp vtdlqatelp gqrvrvswsp vpgatqyrii |
| 541 | vrstqgvert lvlpgsqtaf dlddvgagls ytvrvsarvg pregsasvlt vrrepetpla |
| 601 | vpglrvvvsd atrvrvawgp vpgasgfris wstgsgpess qtlppdstat ditglqpgtt |
| 661 | yqvaysvlrg reegpaaviv artdplgpvr tvhvtqasss svtitwtrvp gatgyrvswh |
| 721 | sahgpeksql vsgeatvael dglepdteyt vhvrahvagv dgppasvvvr tapepvgrvs |
| 781 | rlqilnassd vlritwvgvt gatayrlawg rseggpmrhq ilpgntdsae irgleggvsy |
| 841 | svrvtalvgd regtpvsivv ttppeappal gtlhvvqrge hslrlrwepv praggfllhw |
| 901 | gpegggegsr vlgpelssyh ldglepatqy rvrlsvlgpa gegpsaevta rtesprvpsi |
| 961 | elrvvdtsid svtlawtpvs rassyilswr plrgpgqevp gspqtlpgis ssqrvtglep |
| 1021 | gvsyifsltp vldgvrgpea svtqtpvcpr gladvvflph atqdnahrae atrrvlerlv |
| 1081 | lalgplgpqa vqvgllsysh rpsplfping shdlgiilqr irdmpymdps gnnlgtavvt |
| 1141 | ahrymlapda pgrrqhvpgv mvllvdeplr gdifspirea gasglnvvml gmagadpeql |
| 1201 | rrlapgmdsv qtffavddgp sldqaysgla talcgasftt qprpepcpvy cpkgqkgepg |
| 1261 | emglrgqvgp pgdpglpgrt gapgpqgppg satakgergf pgadgrpgsp gragnpgtpg |
| 1321 | apglkgspgl pgprgdpger gprgpkgepg apgqviggeg pglpgrkgdp gpsgppgprg |
| 1381 | plgdpgprgp pglpgtamkg dkgdrgergp pgpgeggiap gepglpglpg spgpqgpvgp |
| 1441 | pgkkgekgds edgapglpgq pgspgeqgpr gppgaigpkg drgfpgplge agekgergpp |
| 1501 | gpagsrglpg vagrpgakgp egppgptgrq gekgepgrpg dpavvgpava gpkgekgdvg |
| 1561 | pagprgatgv qgergppglv lpgdpgpkgd pgdrgpiglt gragppgdsg ppgekgdpgr |
| 1621 | pgppgpvgpr grdgevgekg degppgdpgl pgkagerglr gapgvrgpvg ekgdqgdpge |
| 1681 | dgrngspgss gpkgdrgepg ppgppgrlvd tgpgarekge pgdrgqegpr gpkgdpglpg |
| 1741 | apgergiegf rgppgpqgdp gvrgpagekg drgppgldgr sgldgkpgaa gpsgpngaag |
| 1801 | kagdpgrdgl pglrgegglp gpsgppglpg kpgedgkpgl ngkngepgdp gedgrkgekg |
| 1861 | dsgasgregr dgpkgergap gilgpqgppg lpgpvgppgq gfpgvpggtg pkgdrgetgs |
| 1921 | kgeqglpger glrgepgsvp nvdrlletag ikasalreiv etwdessgsf lpvperrrgp |
| 1981 | kgdsgeqgpp gkegpigfpg erglkgdrgd pgpqgppgla lgergppgps glagepgkpg |
| 2041 | ipglpgragg vgeagrpger gergekgerg eggrdgppgl pgtpgppgpp gpkvsvdepg |
| 2101 | pglsgeggpp glkgakgepg sngdqgpkgd rgvpgikgdr gepgprgqdg npglpgergm |
| 2161 | agpegkpglq gprgppgpvg ghgdpgppga pglagpagpq gpsglkgepg etgppgrglt |
| 2221 | gptgavglpg ppgpsglvgp qgspglpgqv getgkpgapg rdgasgkdgd rgspgvpgsp |
| 2281 | glpgpvgpkg epgptgapgq avvglpgakg ekgapgglag dlvgepgakg drglpgprge |
| 2341 | kgeagragep gdpgedgqkg apgpkgfkgd pgvgvpgspg ppgppgvkgd lglpglpgap |
| 2401 | gvvgfpgqtg prgemgqpgp sgerglagpp gregipgplg ppgppgsvgp pgasglkgdk |
| 2461 | gdpgvglpgp rgergepgir gedgrpgqeg prgltgppgs rgergekgdv gsaglkgdkg |
| 2521 | dsavilgppg prgakgdmge rgprgldgdk gprgdngdpg dkgskgepgd kgsaglpglr |
| 2581 | gllgpqgqpg aagipgdpgs pgkdgvpgir gekgdvgfmg prglkgergv kgacgldgek |
| 2641 | gdkgeagppg rpglaghkge mgepgvpgqs gapgkeglig pkgdrgfdgq pgpkgdqgek |
| 2701 | gergtpgigg fpgpsgndgs agppgppgsv gprgpeglqg qkgergppge rvvgapgvpg |
| 2761 | apgergeqgr pgpagprgek geaalteddi rgfvrqemsq hcacqgqfia sgsrplpsya |
| 2821 | adtagsqlha vpvlrvshae eeervppedd eyseyseysv eeyqdpeapw dsddpcslpl |
| 2881 | degsctaytl rwyhravtgs teachpfvyg gcggnanrfg treacerrcp prvvqsqgtg |
| 2941 | taqd |

### Age Related Disorders

The disclosure features the treatment of age related disorders. Age related disorders can include but are not limited to, cancer, e.g., skin cancer, e.g., squamous cell carcinoma; melanoma, e.g., superficial spreading melanoma, nodular melanoma, lentigo malignnt melanoma, acral lentiginous melanoma; basal cell carcinoma; or Kaposi's sarcoma; vaginal tearing; chronic or non-healing wounds, dermatoheliosis, wrinkles, lentigod, beborrheic keratoses, diabetes, cardiovascular disease, or nutritional deficiency.

### Indications

The disclosure features a pharmaceutical composition comprising collagen 7, or functional fragment or variant thereof, and one or more pharmaceutically acceptable carriers, for use in treating an age related disorder described herein. The disclosure features a pharmaceutical composition comprising collagen 7, or functional fragment or variant thereof, e.g., described herein, and one or more pharmaceutically acceptable carriers for use in preventing, preventing the progression of, or delaying the onset of one or more symptom associated an age related disorder described herein.

### Subject Selection

Subjects who may benefit from the use of the methods described herein include, but are not limited to, subjects diagnosed with an age related disorder e.g., skin cancer, e.g., squamous cell carcinoma; melanoma, e.g., superficial spreading melanoma, nodular melanoma, lentigo maligna melanoma, acral lentiginous melanoma; basal cell carcinoma; or Kaposi's sarcoma; vaginal tearing; chronic or non-healing wounds. In addition, or alternatively, the subject may have, or may be at risk of developing, an age related disorder e.g., skin cancer, e.g., squamous cell carcinoma; melanoma, e.g., superficial spreading melanoma, nodular melanoma, lentigo maligna melanoma, acral lentiginous melanoma; basal cell carcinoma; or Kaposi's sarcoma; vaginal tearing; chronic or non-healing wounds.

### Organ Transplant Recipients

The disclosure features the treatment of subjects who have received an organ transplant. The organ transplant can be a solid organ transplant e.g., heart, liver, kidney, lung, pancreas, intestine, stomach, testis, etc as contrasted to liquid transplanted tissues, e.g., bone marrow, pancreatic islets, etc.

### Indications

The disclosure features a pharmaceutical composition comprising collagen 7, or functional fragment or variant thereof, and one or more pharmaceutically acceptable carriers, for use in treating a subject who has received an organ transplant as described herein. The disclosure features a pharmaceutical composition comprising collagen 7, or functional fragment or variant thereof, e.g., described herein, and one or more pharmaceutically acceptable carriers for use in preventing, preventing the progression of, or delaying the onset of one or more symptoms associated with receiving an organ transplant as described herein.

### Subject Selection

Subjects who may benefit from the use of the methods described herein include, but are not limited to, subjects who have received an organ transplant. In addition, or alternatively, the subject may have, or may be at risk of developing, a disorder in which the treatment of the disorder is an organ transplant, e.g., cancer of a solid organ, e.g., liver cancer, lung cancer, pancreatic cancer; kidney cancer, testicular cancer, intestinal cancer, stomach cancer, heart cancer; failure of a solid organ, e.g., kidney failure, heart failure, liver failure, lung failure, pancreatic failure; or diagnosed with a disorder which would lead to failure of a solid organ, e.g., chronic renal failure; acute renal failure; type 1 diabetes; type 2 diabetes; chronic kidney disease; immune system conditions, e.g., lupus, chronic viral illnesses, e.g., HIV/AIDS, hepatitis B, hepatitis C; urine blockage in the kidneys; kidney damage; impaired blood flow to the kidneys; congestive heart failure; coronary artery disease; high blood pressure; faulty heart valves; cardiomyopathy; myocarditis; heart arrhythmias; acute heart failure; chronic heart failure; liver failure; liver cirrhosis; biliary duct atresia; cystic fibrosis; early stage liver cancer; primary biliary cirrhosis; primary sclerosing cholangitis; Wilson's disease; lung cancer.

### Preparation of Collagen 7 and Functional Fragments and Variants Thereof

Collagen 7 and functional fragments and variants thereof can be synthesized by standard molecular biology techniques in standard cell lines, e.g., CHO, HEK293, fibroblast or keratinocyte cells. Standard cell culture procedures and conditions may be used for culture of host cells described herein and are known to those skilled in the art. Host cells cultured for expression of recombinant collagen 7, such as HEK293 cells, may be cultured in routinely used cell culture media (e.g. Dulbecco's modified Eagle's medium (DMEM)/Ham's F-12 (1:1) with suitable supplementation of serum, antibiotics, etc, dependent on the application) as referenced in, ((Chen et al. J Bio Chem 277(18): 2118-2124 (2002)), (Chen et al. J Bio Chem 275: 32(11): 24429-24435 (2000)), (Chen et al. J Bio Chem 276(24): 21649-21655 (2001)).

Host cells may be engineered to express other proteins to optimize production of the recombinant collagen 7. This may include, but not limited to, the co-expression of the processing enzymes prolyl hydroxylase, prolidase, or glycosyl-transferase, by exogenously introducing isolated nucleic acid or recombinant expression vectors encoding the appropriate nucleic acid sequence, in host cells comprising collagen 7 nucleic acid sequence or recombinant expression vector. The triple helical assembly of collagen 7 often requires hydroxylation and the presence of ascorbic acid in the host cell growth media. As demonstrated in the reference, (Chen et al. J Bio Chem 277 (18): 2118-2124 (2002)), recombinant type 7 collagen produced, recovered, and purified from HEK293 cells in the presence of ascorbic acid was secreted as an approximately 900-kDa protein, corresponding to the association of three type 7 collagen monomers (each monomer 290-kDa). Ascorbic acid may be used in the host cell culture conditions to aid in proper processing of the recombinant protein.

Suitable vectors for use herein are those that can express collagen 7, prolyl hydroxylase, prolidase, or glycosyl-transferase, or a functional portion thereof. In order to express the proteins described herein, the nucleotide sequence encoding the appropriate protein, or a functional equivalent, can be inserted into a suitable vector. A suitable vector contains the necessary and appropriate transcriptional and translational control sequences for expression of the inserted nucleic acid sequence. Standard methods, known to those skilled in the art, may be used to construct the recombinant expression vectors containing the nucleic acid sequences described herein. These methods include, but are not limited to, in vitro recombinant techniques, synthetic techniques, and in vivo recombination/genetic recombination; the choice of method depends on the nature of the specific nucleotide fragments and may be determined by persons skilled in the art.

Suitable vectors for use herein may contain an origin of replication and a restriction endonuclease sequence site. Persons skilled in the art would have knowledge of suitable origin of replication and restriction endonuclease sequences for use in the host cell. Suitable vectors for use herein may contain sequence elements to aid transcription, including, but not limited to, promoter and enhancer elements. Persons skilled in the art would have knowledge of various transcriptional control elements, including but not limited to, promoters, inducible promoters, and enhancer elements, that would be suitable in the host cell. Suitable vectors for use herein may also contain a selectable marker gene that encodes a product necessary for the host cell to grow and survive under specific conditions, aiding in the selection of host cells into which the vector has been introduced. Typical selection genes may include, but are not limited to, genes encoding a protein that confers resistance to an antibiotic, drug, or toxin (e.g., tetracycline, ampicilin, neomycin, hygromycin, etc). Persons skilled in the art would have knowledge of coding sequences for suitable selectable markers and reporter genes for use in the host cell.

Expression vectors described herein can be introduced into host cells via conventional transformation or transfection techniques. Transformation and transfection techniques include, but are not limited to, calcium phosphate or calcium chloride coprecipitation, DEAE-dextran-mediated transfection, lipofectamine, electroporation, microinjection, and viral mediated transfection (as referenced in U.S. Pat. No. 6,632,637 (McGrew)). Persons skilled in the art would have knowledge of suitable transformation and transfection methods based on the host cell/vector combination. For long term, high yield production of recombinant proteins, stable expression of the recombinant protein may be preferred. Host cells that stably express the recombinant protein may be engineered.

The recombinant expression vectors described herein may be introduced into a suitable host cell, which may include a living cell capable of expressing the protein coding region from the defined recombinant expression vector. The term “host cell” refers not only to the particular subject cell but to the progeny or potential progeny of the particular subject cell. Because certain modifications may occur in succeeding generations due to either mutation or environmental influences, such progeny may not in fact, be identical to the parent cell, but are still included within the scope of the term as used herein. Various host cell expression systems may be utilized to express the nucleic acid molecules described herein. These include, but are not limited to yeast or fungi, transformed with recombinant yeast or fungi expression vectors containing the appropriate nucleic acid sequence; insect cell systems infected with recombinant virus expression vectors or transformed with recombinant plasmid expression vectors containing the appropriate nucleic acid sequence; or mammalian cell systems (e.g., primate cell, human cell, rodent cell, etc) transfected with expression vectors containing the appropriate nucleic acid sequence. Suitable host cells may include primary or transformed cell lines, including, but not limited to, fibroblasts, CHO, HEK293, C127, VERO, BHK, HeLa, COS, MDCK, etc (as referenced in U.S. Pat. No. 6,632,637 (McGrew)). Other suitable host cells are known to those skilled in the art.

Modifications, including, but not limited to, glycosylation, phosphyorylation and processing of protein products may be important to the function of a protein. Different host cells have various characteristics and mechanisms for post-translational processing and modification of proteins. A host cell that is capable of modulating expression of the nucleic acid sequences contained in the vector, or modulating expression of the vector nucleic acid sequences, or modifying and processing the gene product encoded in the vector sequence in a specific manner may be chosen. Mammalian host cells may be chosen to ensure the correct modification and processing of the recombinant protein. Such mammalian host cells may include, but are not limited to, CHO, HEK293, human fibroblasts, and human keratinocytes.

Proteins produced by recombinant methods described herein may be recovered from the host cell culture system according to standard protocols known in the art (e.g., precipitation, centrifugation, etc). Recombinant collagen 7 described herein may be secreted into the host cell medium and recovered by ammonium sulfate precipitation and subsequent centrifugation; as demonstrated in the following reference, (Chen et al. J Bio Chem 277(18): 2118-2124 (2002)). Proteins produced and recovered by recombinant and molecular biology methods described herein, may be purified according to standard protocols known in the art (e.g., dialysis, ion exchange chromatography, affinity chromatography, SDS gel electrophoresis, etc). The recombinant collagen 7 described herein may be purified to homogeneity by ion exchange chromatography; as demonstrated in the following reference, (Chen et al. J Bio Chem 277(18): 2118-2124 (2002)).

Optionally collagen 7 may be further purified. Purification may be achieved using any method known in the art, including, but not limited to affinity chromatography, e.g., an anti-collagen 7 antibody column; hydrophobic interaction chromatography; ion exchange chromatography; size exclusion chromatography; electrophoretic procedures, e.g., isoelectric focusing, differential solubility (e.g., ammonium sulfate precipitation), or extraction, and the like.

### Compositions

The disclosure provides a pharmaceutical composition comprising collagen 7 or functional fragment or variant thereof. Pharmaceutical compositions may take the form of any acceptable pharmaceutical formulation. Pharmaceutical compositions can be formulated in a variety of different forms, such as liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application.

Exemplary pharmaceutical compositions are described below. The pharmaceutical compositions include those suitable for parenteral (including intravenous, subcutaneous, intradermal, intramuscular, and intraarticular), topical (including dermal, transdermal, transmucosal, buccal, sublingual, and intraocular), and rectal administration, although the most suitable route may depend upon, for example, the condition and disorder of the recipient.

Compositions for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The composition may be presented in unit-dose or multi-dose containers, for example sealed ampoules and vials, and may be stored in a freeze-dried (lyophilized) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as EDTA, mannitol, 1,3-butanediol, water, Ringer's solution, an isotonic sodium chloride solution, or other suitable dispersing or wetting and suspending agents. The compositions may contain pharmaceutically acceptable substances or adjuvants, including, but not limited to, EDTA, e.g., 0.5 mM EDTA; pH adjusting and buffering agents and/or tonicity adjusting agents, e.g., sodium acetate, sodium lactate, sodium chloride, potassium chloride, calcium chloride, sorbitan monolaurate; minor amounts of non-toxic auxiliary substances, such as wetting or emulsifying agents or preservatives.

It should be understood that in addition to the ingredients particularly mentioned above, the composition may include other agents conventional in the art having regard to the type of formulation in question.

### Administration

In practicing the methods described herein, collagen 7 or functional fragment or variant thereof may be administered as a single dose. Collagen 7 or functional fragment or variant thereof may be administered as multiple subsequent doses.

In practicing the methods described herein, collagen 7 or functional fragment or variant thereof may be chronically administered. Chronic administration can include the administration of more than one dose of an agent over a period of time. Chronic administration can include regular administration for an extended period of time. Chronic administration can include the administration of therapy over a prolonged period of time, in some cases, for the duration of a subject's lifetime, so that the concentration of the therapeutic agent is maintained at a therapeutically or prophylactically effective level throughout the course of treatment.

The period of time of chronic administration can be for the lifetime of the subject. The period of time of chronic administration can include, but is not limited to, at least 3 months, at least 6 months, at least 1 year, at least 2 years, at least 3 years, at least 4 years, at least 5 years, at least 10 years, at least 15 years, at least 20 years, at least 25 years, at least 30 years, at least 35 years, at least 40 years, at least 45 years, at least 50 years, at least 100 years, at least 150 years, or anytime period between 3 months and 150 years.

Chronic administration can include a series of doses which together provide an effective amount for treating and/or preventing, preventing the progression of, or delaying the onset of symptoms associated with a disorder described herein. A pharmaceutical composition comprising collagen 7 or functional fragment or variant thereof may be administered on various dosing schedules. The dosing schedule can be dependent on several factors including, the severity of the disorder described herein; the specific composition of collagen 7 or functional fragment or variant thereof employed for treatment; the age, body weight, general health, sex and diet of the patient; the time of administration, route of administration, and rate of excretion of the specific composition employed; the duration of the treatment; drugs used in combination or coincidental with the specific collagen 7 or functional fragment or variant thereof composition employed; and like factors well known in the medical arts.

Exemplary dosing schedules of collagen 7 or functional fragment or variant thereof include, once daily, or once weekly, or once every other week, or once monthly, or once every other month, or once every three months, or once every 6 months, or once every 12 months, or once every 18 months, or once every 24 months. The composition can be administered twice per week or twice per month, or once every two, three or four weeks. The composition can be administered as two, three, or more sub-doses at appropriate intervals or even using continuous infusion or delivery through a controlled release formulation. In that case, the therapeutic agent contained in each sub-dose may be correspondingly smaller in order to achieve the total daily dosage. The dosage can also be compounded for delivery over several days, e.g., using a conventional sustained release formulation, which provides sustained release of the agent over a several day period. Sustained release formulations are well known in the art and are particularly useful for delivery of agents at a particular site. The total daily, weekly, or monthly usage of collagen 7 composition can be decided by an attending physician within the scope of sound medical judgment.

The present disclosure features methods including, administering a treatment comprising collagen 7 or functional fragment or variant thereof. The disclosure can further include selecting a regimen, e.g., dosage, formulation, route of administration, number of dosages, or adjunctive or combination treatments of collagen 7 or functional fragment or variant thereof. The disclosure can further include the administration of the selected regimen.

A treatment comprising collagen 7 or functional fragment or variant thereof disclosed herein, may be administered by any route, including by those routes currently accepted and approved for known products. Exemplary routes of administration include, e.g., topical (e.g. by powders, ointments, creams, gels, lotions, and/or drops) or systemic (e.g., intravenous).

Even upon improvement of a patient's condition, chronic administration of collagen 7 or functional fragment or variant thereof may be administered. The dosage or frequency of administration, or both, may not be reduced, as a function of disease symptoms. The dosage or frequency of administration, or both, may be reduced, as a function of disease symptoms, to a level at which the improved condition is retained. Subjects may require intermittent changes in dosage or frequency of administration of treatment on a long-term basis upon any recurrence of disease symptoms.

### Combination Treatments

The present disclosure encompasses combined administration of an additional agent or agents with collagen 7 or functional fragments and variants thereof. Additional agents may include, but are not limited to, chemotherapeutic agents, immunosuppressants, antibiotics, analgesics, opioids, anti-virals, or anti-inflammatory agents. Combination therapy can also include but is not limited to surgery, e.g., Mohs surgery, cryosurgery, curettage, electrodissection, laser surgery, demabrasion, excision, e.g., simple excision, shave excision; photodynamic therapy; radiation therapy; chemotherapy; biologic therapy, e.g. interferon and imiquimod.

Additional agents disclosed herein, may be administered by any route, including by those routes currently accepted and approved for known products. Exemplary routes of administration include, e.g., topical (e.g. by powders, ointments, creams, gels, lotions, and/or drops) and systemic (e.g., intravenous).

### Chemotherapeutic Agents

Chemotherapeutic agents can be administered systemically, e.g., intravenously, or topically, e.g., in the form of a cream, lotion, or ointment, e.g., in the form of a cream, lotion, or ointment applied to the skin or skin lesion.

Chemotherapeutic agents can include but are not limited to, antimetabolites (e.g., folic acid, purine, and pyrimidine derivatives) and alkylating agents (e.g., nitrogen mustards, nitrosoureas, platinum, alkyl sulfonates, hydrazines, triazenes, aziridines, spindle poison, cytotoxic agents, toposimerase inhibitors and others). Exemplary agents include aclarubicin, actinomycin, alitretinon, altretamine, aminopterin, aminolevulinic acid, amrubicin, amsacrine, anagrelide, arsenic trioxide, asparaginase, atrasentan, belotecan, bexarotene, endamustine, bleomycin, bortezomib, busulfan, camptothecin, capecitabine, carboplatin, carboquone, carmofur, carmustine, celecoxib, chlorambucil, chlormethine, cisplatin, cladribine, clofarabine, crisantaspase, cyclophosphamide, cytarabine, dacarbazine, dactinomycin, daunorubicin, decitabine, demecolcine, docetaxel, doxorubicin, efaproxiral, elesclomol, elsamitrucin, enocitabine, epirubicin, estramustine, etoglucid, etoposide, floxuridine, fludarabine, fluorouracil (5fu), fotemustine, gemcitabine, gliadel implants, hydroxycarbamide, hydroxyurea, idarubicin, ifosfamide, irinotecan, irofulven, ixabepilone, larotaxel, leucovorin, liposomal doxorubicin, liposomal daunorubicin, lonidamine, lomustine, lucanthone, mannosulfan, masoprocol, melphalan, mercaptopurine, mesna, methotrexate, methyl aminolevulinate, mitobronitol, mitoguazone, mitotane, mitomycin, mitoxantrone, nedaplatin, nimustine, oblimersen, omacetaxine, ortataxel, oxaliplatin, paclitaxel, pegaspargase, pemetrexed, pentostatin, pirarubicin, pixantrone, plicamycin, porfimer sodium, prednimustine, procarbazine, raltitrexed, ranimustine, rubitecan, sapacitabine, semustine, sitimagene ceradenovec, strataplatin, streptozocin, talaporfin, tegafur-uracil, temoporfin, temozolomide, teniposide, tesetaxel, testolactone, tetranitrate, thiotepa, tiazofurine, tioguanine, tipifarnib, topotecan, trabectedin, triaziquone, triethylenemelamine, triplatin, tretinoin, treosulfan, trofosfamide, uramustine, valrubicin, verteporfin, vinblastine, vincristine, vindesine, vinflunine, vinorelbine, vorinostat, zorubicin, and other cytostatic or cytotoxic agents described herein. Additional exemplary chemotherapeutic agents, include 5-fluorouracil (5fu), adrucil (fluorouracil), aldara (imiquimod), efudex (fluorouracil), erivedge (vismodegib), fluoroplex (fluorouracil), fluorouracil, imiquimod, vismodegib, aldesleukin, dacarbazine, dtic-dome (dacarbazine), ipilimumab, proleukin (aldesleukin), vemurafenib, yervoy (ipilimumab), zelboraf (vemurafenib), retinoids.

### Targeted Therapy

Collagen 7 or functional fragments and variants thereof described herein, can be administered with a targeted cancer therapy. Targeted therapy constitutes the use of agents specific for the deregulated proteins of cancer cells. An exemplary targeted therapy includes vismodegib (erivedge). Small molecule targeted therapy drugs are generally inhibitors of enzymatic domains on mutated, overexpressed, or otherwise critical proteins within the cancer cell. Prominent examples are the tyrosine kinase inhibitors such as axitinib, bosutinib, cediranib, desatinib, erlotinib, imatinib, gefitinib, lapatinib, lestaurtinib, nilotinib, semaxanib, sorafenib, sunitinib, and vandetanib, and also cyclin-depdendent kinase inhibitors such as alvocidib and seliciclib. Monoclonal antibody therapy is another strategy in which the therapeutic agent is an antibody which specifically binds to a protein on the surface of the cancer cells. Examples include the anti-HER2/neu antibody trastuzumab (HERCEPTIN®) typically used in breast cancer, and the anti-CD20 antibody rituximab and Tositumomab typically used in a variety of B-cell malignancies. Other exemplary antibodies include cetuximab, panitumumab, trastuzumab, alemtuzumab, bevacizumab, edrecolomab, gemtuzumab, ranibizumab. exemplary fusion proteins include aflibercept and denileukin diftitox. In some embodiments, the targeted therapy can be used in combination with Collagen 7 or functional fragments and variants thereof described herein.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell. An example of such therapy includes BEXXAR®.

### Immunotherapy

Collagen 7 or functional fragments and variants thereof described herein, can be administered with an immunotherapy. Cancer immunotherapy refers to a diverse set of therapeutic strategies designed to induce the patient's own immune system to fight the tumor. Contemporary methods for generating an immune response against tumors include intravesicular Bacille Calmette-Guerin (BCG) vaccine immunotherapy, and use of interferons and other cytokines to induce an immune response. Allogeneic hematopoietic stem cell transplantation can be considered a form of immunotherapy, since the donor's immune cells will often attack the tumor in a graft-versus-tumor effect. In some embodiments, the immunotherapy agents can be used in combination with Collagen 7 or functional fragments and variants thereof described herein. Exemplary immunotherapy includes injections of BCG vaccine, interleukin-2, interferon, ipilimumab (Yervoy); vemurafenib (Zelboraf).

### Hormonal Therapy

Collagen 7 or functional fragments and variants thereof described herein, can be administered with a hormonal therapy. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial. The hormonal therapy agents can be used in combination with Collagen 7 or functional fragments and variants thereof described herein.

### Anti-Inflammatory Agents

Collagen 7 or functional fragments and variants thereof described herein, can be administered with an anti-inflammatory agent. Anti-inflammatory agents can include, but are not limited to, non-steroidal anti-inflammatory agents (e.g., salicylates (aspirin (acetylsalicylic acid), diflunisal, salsalate), propionic acid derivatives (ibuprofen, naproxen, fenoprofen, ketoprofen, flurbiprofen, oxaprozin, loxoprofen), acetic acid derivatives (indomethacin, sulindac, etodolac, ketorolac, diclofenac, nabumetone), enolic acid (oxicam) derivatives (piroxicam, meloxicam, tenoxicam, droxicam, lornoxicam, isoxicam), fenamic acid derivatives (fenamates)(mefenamic acid, meclofenamic acid, flufenamic acid, tolfenamic acid), selective cox-2 inhibitors (coxibs) (celecoxib), sulphonanilides (nimesulide). Steroids (e.g. hydrocortisone (cortisol), cortisone acetate, prednisone, prednisolone, methylprednisolone, dexamethasone, betamethasone, triamcinolone, beclometasone, fludrocortisone acetate, deoxycorticosterone acetate, aldosterone).

### Analgesic Agents

Analgesics can include but are not limited to, opiates (e.g. morphine, codeine, oxycodone, hydrocodone, dihydromorphine, pethidine, buprenorphine, tramadol, venlafaxine), paracetomal and Non-steroidal anti-inflammatory agents (e.g., salicylates (aspirin (acetylsalicylic acid), diflunisal, salsalate), propionic acid derivatives (ibuprofen, naproxen, fenoprofen, ketoprofen, flurbiprofen, oxaprozin, loxoprofen), acetic acid derivatives (indomethacin, sulindac, etodolac, ketorolac, diclofenac, nabumetone), enolic acid (oxicam) derivatives (piroxicam, meloxicam, tenoxicam, droxicam, lornoxicam, isoxicam), fenamic acid derivatives (fenamates)(mefenamic acid, meclofenamic acid, flufenamic acid, tolfenamic acid), selective cox-2 inhibitors (coxibs) (celecoxib), sulphonanilides (nimesulide).

### Antiemetic Agents

Collagen 7 or functional fragments and variants thereof described herein, can be administered with an antiemetic agent. Antiemetic agents can include, but are not limited to, 5-HT3 receptor antagonists (dolasetron (anzemet), granisetron (kytril, sancuso), ondansetron (zofran), tropisetron (navoban), palonosetron (aloxi), mirtazapine (remeron)), dopamine antagonists (domperidone, olanzapine, droperidol, haloperidol, chlorpromazine, promethazine, prochlorperazine, metoclopramide (reglan), alizapride, prochlorperazine (compazine, stemzine, buccastem, stemetil, phenotil), NK1 receptor antagonist (aprepitant (emend), antihistamines (cyclizine, diphenhydramine (benadryl), dimenhydrinate (gravol, dramamine), meclozine (bonine, antivert), promethazine (pentazine, phenergan, promacot), hydroxyzine), benzodiazapines (lorazepam, midazolam), anticholinergics (hyoscine), steroids (dexamethasone).

### Immunosuppressants

Immunosuppressants can include but are not limited to cyclosporine A (CyA, neoral, gengraf, sangcya, sandimmune); prograf (tacrolimus); imuran (azathioprine); steroids, e.g., prednisone, deltasone, methylprednisolone; or rapamune (sirolimus).

### Antibiotics

Antibiotics can include, but are not limited to, aknilox, ambisome, amoxycillin, ampicillin, augmentin, avelox, azithromycin, bactroban, betadine, betnovate, blephamide, cancidas, cefaclor, cefadroxil, cefdinir, cefepime, cefix, cefixime, cefoxitin, cefpodoxime, cefprozil, cefuroxime, cefzil, cephalexin, cephazolin, ceptaz, chloramphenicol, chlorhexidine, chloromycetin, chlorsig, ciprofloxacin, clarithromycin, clindagel, clindamycin, clindatech, cloxacillin, colistin, co-trimoxazole, demeclocycline, diclocil, dicloxacillin, doxycycline, duricef, erythromycin, flagyl alcohol, flagyl dosage, flagyl pregnancy, flagyl side effects, flagyl treatment, flamazine, floxin, framycetin, fucidin, furadantin, fusidic, gatifloxacin, gemifloxacin, gemifloxacin, ilosone, iodine, levaquin, levofloxacin, loceryl, lomefloxacin, maxaquin, mefoxin, meronem, minocycline, moxifloxacin, myambutol, mycostatin neosporin, netromycin, nitrofurantoin, norfloxacin, norilet, ofloxacin, omnicef, ospamox, oxytetracycline, paraxin, penicillin, pneumovax, polyfax, povidone, rifadin, rifampin, rifaximin, rifinah, rimactane, rocephin, roxithromycin, seromycin, soframycin, sparfloxacin, staphlex, targocid, tetracycline, tetradox, tetralysal, tobramycin, tobramycin, trecator, tygacil, vancocin, velosef, vibramycin, xifaxan, zagam, zitrotek, zoderm, zymar, and zyvox.

### Anti-Viral Agents

Anti-viral agents can include, but are not limited to, abacavir, aciclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, integrase inhibitor, interferon type iii, interferon type ii, interferon type i, interferon, lamivudine, lopinavir, loviride, maraviroc, moroxydine, methisazone, nelfinavir, nevirapine, nexavir, nucleoside analogues, oseltamivir, peginterferon alfa-2a, penciclovir, peramivir, pleconarilpodophyllotoxin, protease inhibitor, raltegravir, reverse transcriptase inhibitor, ribavirin, rimantadine, ritonavir, pyramidine, saquinavir, stavudine, tea tree oil, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

When collagen 7 or functional fragment or variant thereof is administered in combination with an additional agent or a plurality of agents, the dosage of collagen 7 or functional fragment or variant thereof may on its own comprise an effective amount and an additional agent or agents may further augment the therapeutic benefit to the patient. Alternatively the combination of collagen 7 or functional fragment or variant thereof and a second agent may together comprise an effective amount for treating and/or preventing, preventing a disorder described herein. The effective amounts may be defined in the context of particular treatment regimens, including, e.g., timing and number of administrations, modes of administrations, formulations, etc.

What is claimed is:

1. A method for treating a chronic, non-healing skin condition in a subject comprising administering to the subject an effective amount of a pharmaceutical composition comprising collagen 7, or functional fragments and variants thereof, wherein the subject is an elderly subject.

2. The method of claim 1, wherein the chronic, non-healing skin condition is associated with an age related disorder.

3. The method of claim 2, wherein the age related disorder is a skin cancer, a chronic skin lesion, a benign skin lesion, diabetes, mucosal tissue tearing, vaginal tearing, anal tearing, a chronic or non-healing wound, dermatoheliosis, wrinkles, lentigod, beborrheic keratoses, cardiovascular disease, nutritional deficiency, or a disorder in which the treatment of the disorder is an organ transplant.

4. The method of claim 3, wherein the age related disorder is a skin cancer.

5. The method of claim 4, wherein the skin cancer has not been surgically excised.

6. The method of claim 4, wherein the skin cancer has been surgically excised.

7. The method of claim 1, wherein the subject is at least 60 years of age.

8. The method of claim 1, wherein the administration is chronic administration.

9. The method of claim 8, wherein the administration is through intravenous injection or topical application.

10. The method of claim 8, wherein the pharmaceutical composition comprising collagen 7 is administered in combination with one or more additional therapies.

11. The method of claim 10, wherein the additional therapies are one or more selected from the group comprising chemotherapeutic agents, targeted cancer therapies, immunotherapy, hormonal therapy, anti-inflammatory agents, analgesic agents, opioids, moisturizers, antidepressants, anti-osteoporosis agents, antiemetic agents, immunosuppressants, antibiotics, and anti-viral agents.

12. A method for preventing, preventing the progression of, or delaying the onset of a skin wound in a subject, comprising administering to the subject an effective amount of a pharmaceutical composition comprising collagen 7, or functional variants thereof, wherein the subject is at risk for developing a chronic, non-healing skin condition.
